(19) United States
(12) Patent Application Publication
(10) Pub. No.: US 2017/0215698 A1
(43) Pub. Date: Aug. 3, 2017

(54) SYSTEM AND METHOD FOR PROVIDING USER FEEDBACK INDICATIONS DURING INTRA-ORAL SCANNING PROCESS

(71) Applicant: DENTAL WINGS INC., MONTREAL (CA)

(72) Inventor: MICHAEL RYNERSON, MONTREAL (CA)

(21) Appl. No.: 15/417,866

(22) Filed: Jan. 27, 2017

Related U.S. Application Data
(60) Provisional application No. 62/288,083, filed on Jan. 28, 2016.

(51) Int. Cl. A61B 1/00; H04N 13/04; A61C 9/00; A61B 1/24; A61B 5/01; H04N 13/02; A61B 1/06

(52) U.S. Cl. CPC A61B 1/00055; H04N 13/0221; H04N 13/0253; H04N 13/0275; H04N 13/044; A61B 1/00048; A61B 1/0607; A61B 1/24; A61B 5/01; A61C 9/0053; A61B 1/00009; A61B 2562/0271

(57) ABSTRACT

An intra-oral scanning system includes a hand piece having a scanning head with one or more projectors for projecting light onto an oral region and image sensors for detecting reflected light in the oral region. A computer system is operatively connected to the hand piece for receiving data from the image sensors. The data is representative of a scanned area in the oral region. The computer system images a 3D representation of the scanned area and a display assembly operatively connected to the computer system displays the 3D representation of the scanned area. A transducer operatively connected to the computer system emits a visual, audible and/or vibratory signal that provides information related to a status of the scanning system.

### CROSS REFERENCE TO RELATED APPLICATIONS

This application claims benefit under 35 U.S.C. §119(e) of U.S. provisional application Ser. No. 62/288,083 filed on Jan. 28, 2016 which is incorporated herein in its entirety by reference.

### TECHNICAL FIELD

This invention relates generally to intra-oral scanning processes for obtaining a scan of a patient's teeth that may be used in dental treatment procedures, including dental appliances. More specifically, this invention relates to feedback indications provided to an operator during an intra-oral scanning process using a dental appliance.

### BACKGROUND TO THE INVENTION

Devices for scanning intra-oral region, such as a plurality of teeth or a dental cast to provide a 3D scan are known already.

Current intra-oral scanning devices require the user to look at a monitor during the scanning process to assess if data is being captured appropriately. Forcing the operator to look away from the oral cavity during the scan process can lead to problems and inefficiencies.

There is therefore a need for more effective intra-oral scanning device that allows the operator of an intra-oral scanner to receive all or most of the information needed to complete the scanning portion of the process without needing to look at a monitor.

### SUMMARY OF THE INVENTION

In an aspect there is provided an intra-oral scanning system comprising: a hand piece comprising a scanning head with one or more projectors for projecting light onto an oral region and image sensors for detecting reflected light in said oral region; a computer system operatively connected to said hand piece for receiving data from said image sensors, said data being representative of a scanned area in said oral region, said computer system for imaging a 3D representation of said scanned area; a display assembly operatively connected to said computer system for displaying said 3D representation of said scanned area; a transducer operatively connected to said computer system for emitting a visual, audible and/or tactile signal such as a vibration or knock or the like that provides information related to a status of said scanning system.

In another aspect there is provided a method for intra-oral scanning, comprising: scanning an oral region by means of a hand piece comprising a scanning head with one or more projectors for projecting light onto an oral region and image sensors for detecting reflected light in said oral region; receiving data from said image sensors at a computer system, said data being representative of a scanned area in said oral region; imaging a 3D representation of said scanned area by means of said data and computer system; displaying said 3D representation of said scanned area by means of a display assembly operatively connected to said computer system; emitting a visual, audible and/or tactile signal by means of a transducer operatively connected said computer system for providing information related to said scanning or said receiving.

A preferred advantage of the present invention is that the operator of the intra-oral scanner system receives all or most of the information needed to properly complete the scanning portion of the process without needing to look at a monitor or to look away from the oral cavity during the scanning procedure.

### BRIEF DESCRIPTION OF THE DRAWINGS

The invention will be better understood by way of the following detailed description of embodiments of the invention with reference to the appended drawings, in which:

FIG. 1 is a perspective view of an intra-oral scanning system, according to an embodiment of the invention;

FIG. 2 is a perspective view of an intra-oral scanning hand piece, according to an embodiment of the invention;

FIG. 3 is a front plan view of the intra-oral scanning hand piece shown in FIG. 2;

FIG. 4 is a rear plan view of the intra-oral scanning hand piece shown in FIG. 2;

FIGS. 5 and 6 are respective left and right side plan views of the intra-oral scanning hand piece shown in FIG. 2;

FIG. 7 is a top plan view of the intra-oral scanning hand piece shown in FIG. 2;

FIG. 8 is a front plan view of a wearable goggles used in another embodiment of the present invention;

FIG. 9 is a block diagram of a system for intra-oral scanning, according to an embodiment of the present invention; and

FIG. 10 is a flowchart of a method for intra-oral scanning, according to an embodiment of the present invention.

### DETAILED DESCRIPTION OF THE ILLUSTRATIVE EMBODIMENTS

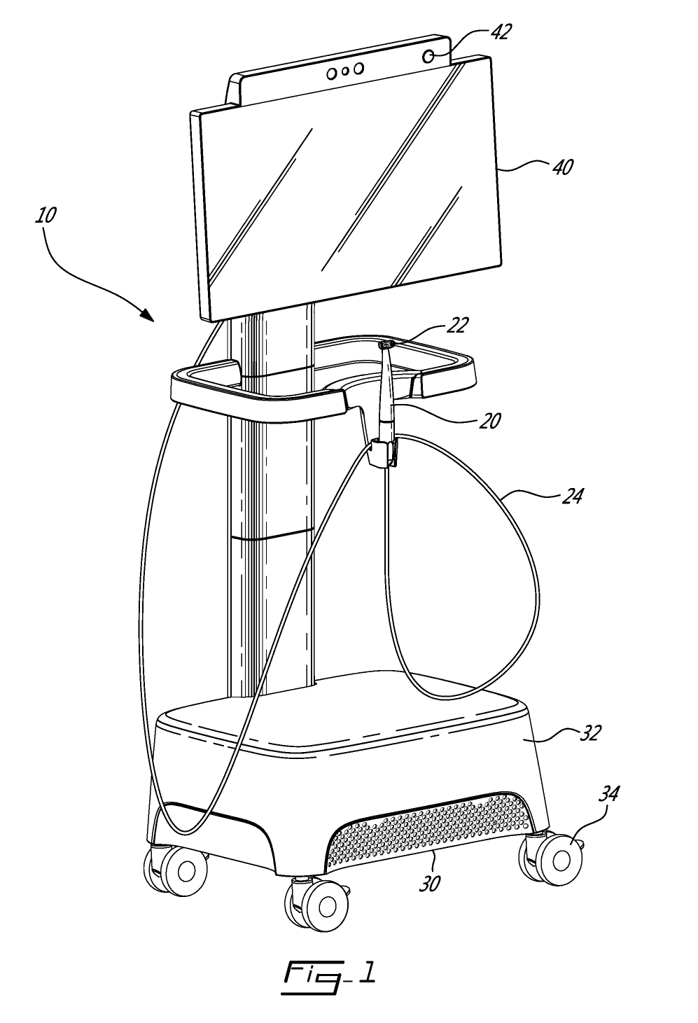

Referring now to FIGS. 1 and 9, there is shown an intra-oral scanning system 10 according to a preferred embodiment of the present invention. The system 10 includes a hand piece 20 that is to be manipulated by an operator, such as a dentist or technician, while performing the scanning of an oral region 80.

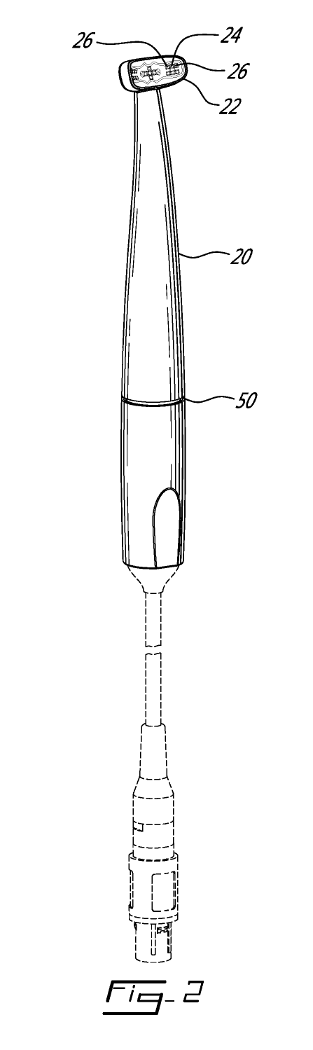

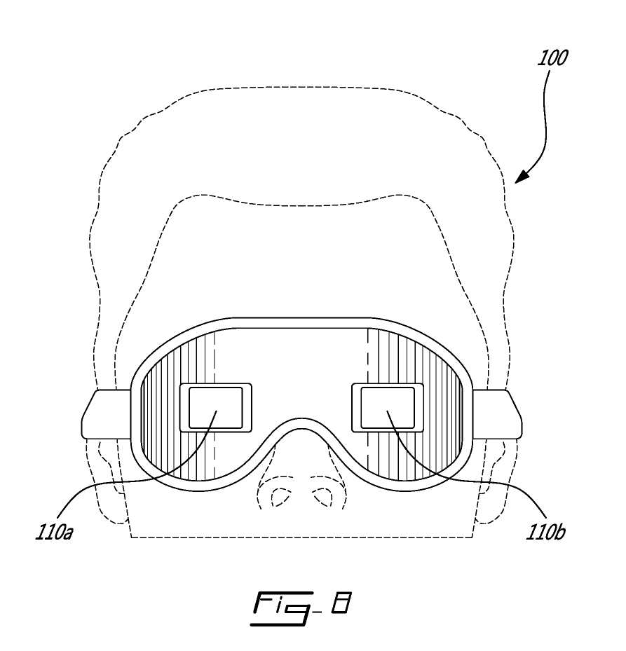

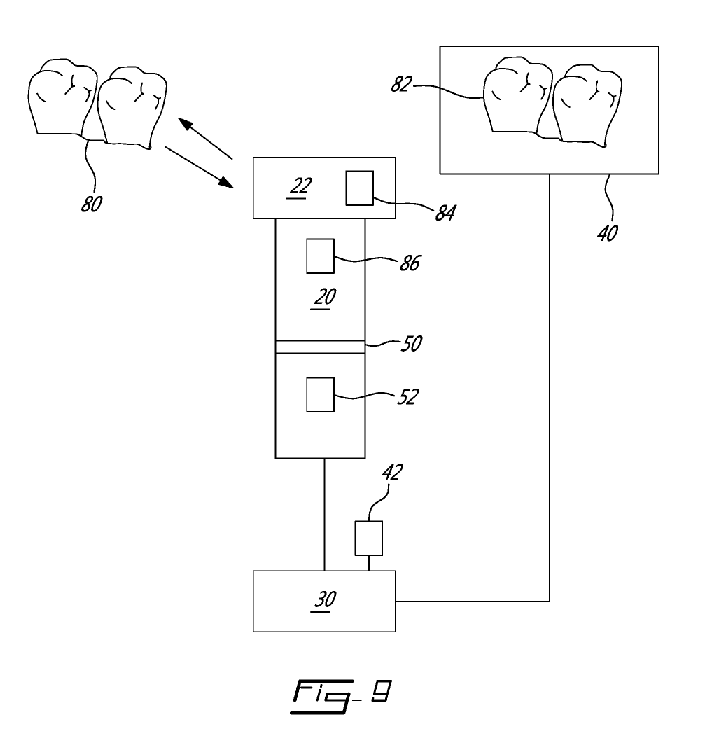

Referring now to FIGS. 2 to 7, the hand piece 20 includes a scanning head 22 that is preferably curved inwards with respect to the oral region 80 to be scanned. As best shown in FIGS. and 2 and 3, the scanning head 22 has a plurality of projectors 24 for projecting light onto the oral region 80 and image sensors 26 for detecting reflected light in the oral region 80. The projectors 24 may include LED lights or other suitable light sources and the image sensors 26 may be CMOS sensors. The purpose of the projectors 24 and image sensors 26 is to generate stereoscopic images of the oral region 80.

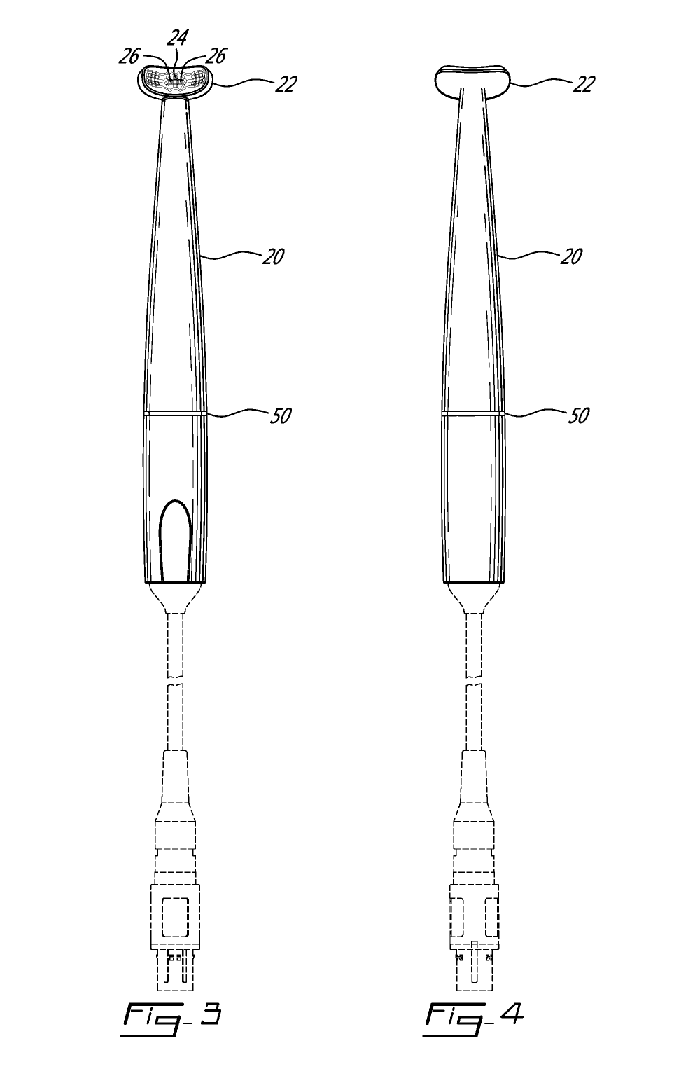

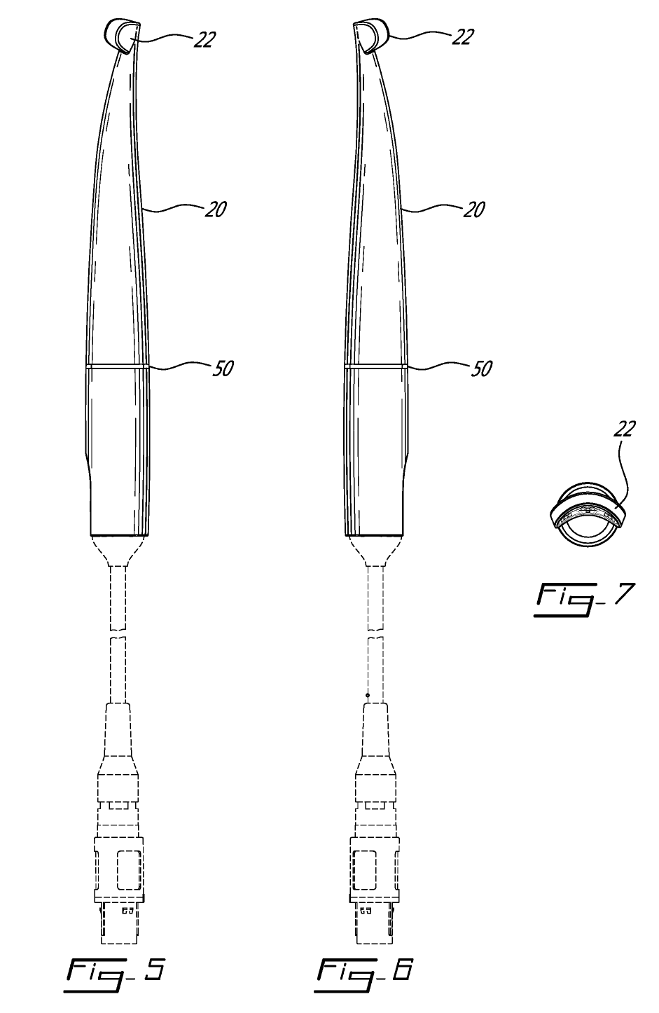

Referring back to FIGS. 1 and 9, a computer system 30 is operatively connected to the hand piece 20 for receiving data from the image sensors 26 (shown in FIGS. 2 and 3). The hand piece 20 is shown operatively connected to the computer system 30 via a communication cable 24, which also provides electric power to the hand piece 20. Of course, it is possible that communication from the hand piece 20 to the computer system 30 be wireless, but this would require a separate power source in the hand piece 20 such as a rechargeable battery, which is more expensive.

Still referring to FIGS. 1 and 9, the computer system 30 is shown mounted inside on a cart comprising a base 32 that is provided with wheels 34. During operation of the system 10, the scanning head 22 is directed toward the oral region 80 of a patient, light is projected in the oral region 80 by the projectors 24 and light reflected is captured by the image sensors 26. The data captured by the image sensors 26 is representative of a scanned area in the oral region 80. The computer system 30 images a 3D representation of the scanned area. A display assembly 40, such as a monitor, that is operatively connected to the computer system 30 displays the 3D representation 82 of the scanned area. A transducer is operatively connected to the computer system 30 for emitting a visual, audible and/or tactile signal such as a vibration or knock or the like that provides information related to a status of the scanning system 10.

In one embodiment and referring back to FIGS. 2, 3 and 9, the transducer may include a one or more sets of lights 50 or illumination sources that are mounted on the hand piece 20. The lights 50 may define a ring in a middle portion of the hand piece 20. These lights 50 or illumination sources may turn on or off automatically depending on a status of the scanning system 10. The illumination sources may project the light intra-orally or extra-orally with respect to the oral region 80 to inform the operator of the hand piece 20 about a status of the system 10. The illumination sources or lights 50 may change colour depending on the status of the scanning system 10. For example, the lights 50 may be red if the system 10 is not ready to operate properly and the lights 50 may change to green when the system 10 is ready and operational. In general, the information provided by the lights 50 may provide an indication to the operator that the system 10 or any part of the system 10 is on or off. The lights 50 may also provide an indication that the scanning head 22 is in an appropriate or optimal location relative to the patient's teeth for carrying out scanning. The light 50 may also provide an indication that the system 10 is capturing data and that the data is of an appropriate quality or that the system 10 is operating nominally, or that the handle piece 20 or scanning head 22 is overheating. The lights 50 may also provide an indication that the handle piece 20 or scanning head 22 is damaged. The lights 50 may also indicate that the handle piece 20 is being moved too quickly or too slowly with respect to a predetermined operational velocity of the scanning head 22. The lights 50 may also indicate that the scanning head 22 needs to be moved closer or further away from the teeth. The lights 50 may also indicate that the operator needs to refer to the monitor for further information.

In another embodiment and referring back to FIGS. 3 and 9, the transducer may include a tactile feedback device 52 mounted on the hand piece 20 for emitting a tactile feedback signal such as a vibration that may be sensed by the operator of the hand piece 20. The tactile feedback device 52 could also be located on a foot pedal used by the operator of the hand piece 20 or on any other object in contact with the operator of the hand piece 20. Again, the tactile feedback device 52 may provide the operator with similar indications as the lights 50, as explained above.

In another embodiment and referring back to FIGS. 1 and 9, the transducer may include a speaker 42 for emitting an audible signal. The speaker 42 may be part of the computer system 30 or the display assembly 40 or mounted on the hand piece 20 or may be located in any other convenient location so that it can be heard by the operator of the hand piece 20. The speaker 42 may audibly inform the operator of the handle piece 20 of different information related to the scanning procedures. Again, the speaker 42 may inform the operator about similar indications as the lights 50, as explained above. For example, the speaker 42 may emit a voiced message indicating that the operator needs to refer to the display assembly 40 or monitor for further information.

Referring now to FIG. 7, there is shown a set of wearable glasses 100 or googles that may be used by the operator of the hand piece 20. In this alterative embodiment, the display assembly 40 instead of being a monitor may be incorporated into the wearable glasses 100. The wearable glasses have two see-through screens 110a, 110b that transmit light such that the operator can directly see the real environment in front of his/her eyes through the see-through screens 110a, 110b. An image of the scanned region is projected on each see-through screen 110a, 110b. The images provided on the right and the left eye corresponds to a graphical representation of the scanned region in stereoscopy such that the scanned region appears in three-dimensions to the operator of the hand piece 20. Such wearable glasses 100 may be similar to those disclosed in WO2008/092230 which is incorporated herein by reference in its entirety.

Referring back to FIG. 9, the scanning head 22 includes a temperature sensor 84 for detecting a temperature of the oral region 80. This sensor 84 is useful for preventing the scanning head 22 from becoming steamed up by water vapor that is present inside the oral region 80 and which otherwise might condense on the scanning head 22. As the system 10 is turned on, the scanning head 22 is automatically heated to a temperature ranging from about 30 to 40 degrees Celsius, which is monitored by the temperature sensor 84. Once the temperature in the scanning head 22 has reached a predetermined temperature, the operator may be informed via one of the transducers. The hand piece 20 may also include another temperature sensor 86 to monitor the temperature inside the hand piece 20. As the hand piece 20 is used over several minutes by the operator, the hand piece 20 may overheat and the operator may be informed of this situation by means of one the transducers, as explained above.

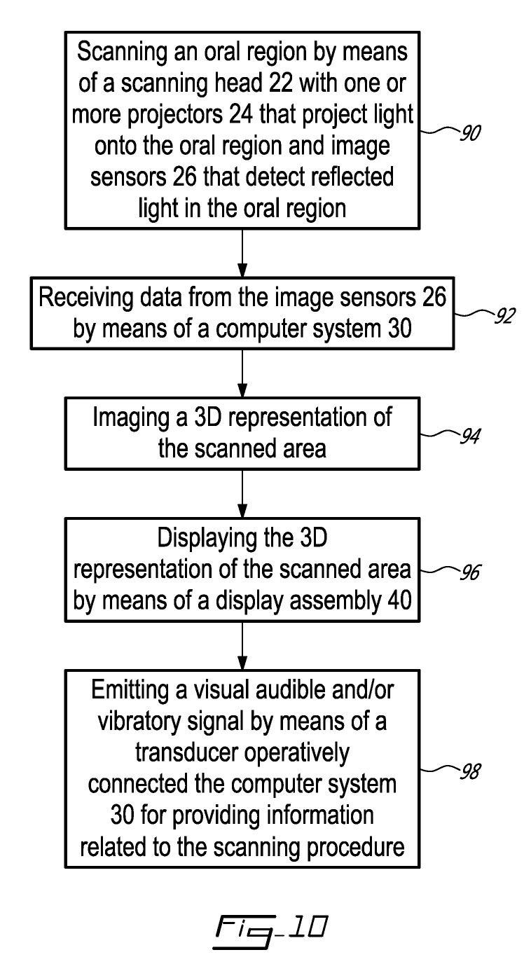

Referring now to FIG. 10 and back to FIGS. 1 and 9, there is shown a flowchart of a method for intra-oral scanning, according to an embodiment of the present invention. The method begins by scanning 90 an oral region by means of the hand piece 20 with the scanning head 22 including the projectors 24 for projecting light onto the oral region 80 and image sensors 26 for detecting reflected light in the oral region. The method includes receiving 92 data from the image sensors 26 at the computer system 30. The data is representative of a scanned area in the oral region 80. The method includes imaging 94 a 3D representation of the scanned area by means of the data and computer system 30. The method includes displaying 96 the 3D representation of the scanned area by means of the display assembly 40 that is operatively connected to the computer system 30. The method includes emitting 98 a visual, audible and/or tactile feedback signal by means of a transducer operatively connected the computer system 30 for providing information related to said scanning 90 or said receiving 92.

As explained above, an advantage of the present invention is that the operator of the intra-oral scanner system receives all or most of the information needed to properly complete the scanning portion of the process without needing to look at a monitor or to look away from the oral cavity during the scanning procedure

Although the present invention has been described hereinabove by way of specific embodiments thereof, it can be modified, without departing from the spirit and nature of the subject invention as defined in the appended claims.

1. An intra-oral scanning system comprising:
a hand piece comprising a scanning head with one or more projectors for projecting light onto an oral region and image sensors for detecting reflected light in said oral region;
a computer system operatively connected to said hand piece for receiving data from said image sensors, said data being representative of a scanned area in said oral region, said computer system for imaging a 3D representation of said scanned area;
a display assembly operatively connected to said computer system for displaying said 3D representation of said scanned area; and
a transducer operatively connected to said computer system for emitting at least one of a visual, audible and tactile signal that provides information related to a status of said scanning system.

2. The intra-oral scanning system of claim 1, wherein said transducer comprises one or more lights.

3. The intra-oral scanning system of claim 2, wherein said one or more lights are mounted on said hand piece.

4. The intra-oral scanning system of claim 2, wherein said one or more lights define a ring around said hand piece.

5. The intra-oral scanning system of claim 1, wherein said transducer comprises one or more illumination sources that turn on or off depending on said status of said scanning system.

6. The intra-oral scanning system of claim 1, wherein said one or more illumination sources are projected intra-orally or extra-orally with respect to said oral region.

7. The intra-oral scanning system of claim 1, wherein said transducer comprises one or more illumination sources that change colour depending on the status of said scanning system.

8. The intra-oral scanning system of claim 1, wherein said tactile signal is a vibration and said transducer comprises a vibration device for emitting said vibration.

9. The intra-oral scanning system of claim 8, wherein said tactile device is located on said hand piece.

10. The intra-oral scanning system of claim 1, wherein said transducer comprises a speaker for emitting said audible signal.

11. The intra-oral scanning system of claim 1, wherein said display assembly is mounted on wearable glasses.

12. The intra-oral scanning system of claim 1, wherein said scanning head comprises a temperature sensor for detecting a temperature of said oral region.

13. The intra-oral scanning system of claim 1, wherein said hand piece comprises a temperature sensor for detecting a temperature of said hand piece.

14. The intra-oral scanning system of claim 1, wherein said status corresponds to one of said scanning system being one of on and off, said scanning head being in an operational position relative to said oral region, said computer system receiving said data from said image sensors, said computer system receiving said data from said image sensors that is of a predetermined quality, said scanning system operating nominally, said hand piece overheating and said hand piece being damaged.

15. The intra-oral scanning system of claim 1, wherein using said data from said image sensors said computer system senses a speed of movement of said hand piece, and wherein said status corresponds to said sensed speed of movement being one of too quick and too slow with respect to a predetermined range of operational velocities.

16. The intra-oral scanning system of claim 1, wherein using said data from said image sensors said computer system senses a distance of said hand piece to said scanning area, and wherein said status corresponds to said sensed distance of said hand piece being one of positioned too far and too close to said scanning area with respect to a predetermined range of operational distances.

17. The intra-oral scanning system of claim 1, wherein said status corresponds to a voiced message indicating that an operator needs to refer to the display assembly for further information.

18. A method for intra-oral scanning, comprising:
scanning an oral region by means of a hand piece comprising a scanning head with one or more projectors for projecting light onto an oral region and image sensors for detecting reflected light in said oral region;
receiving data from said image sensors at a computer system, said data being representative of a scanned area in said oral region;
imaging a 3D representation of said scanned area by means of said data and computer system;
displaying said 3D representation of said scanned area by means of a display assembly operatively connected to said computer system; and
emitting at least one of a visual, audible and tactile signal by means of a transducer operatively connected to said computer system for providing information related to said scanning or said receiving.

19. The method of claim 18, further comprising determining from said data a speed of movement of said hand piece, comparing said speed of movement with a predetermined range of operational velocities, and wherein said information related to said scanning comprises said speed of movement being one of too quick and too slow with respect to said predetermined range of operational velocities.

20. The method of claim 18, further comprising determining from said data a distance of said hand piece from said scanning area, comparing said distance with a predetermined range of operational distances, and wherein said information related to said scanning comprises said distance being one of too close to said scanning area and too far from said scanning area with respect to said predetermined range of operational distances.
